(19) United States
(12) Patent Application Publication
(10) Pub. No.: US 2024/0013909 A1
(43) Pub. Date: Jan. 11, 2024

(54) METHOD AND SYSTEM FOR WIRED AND WIRELESS COMMUNICATION USING ULTRASOUND DEVICES

(71) Applicant: BFLY OPERATIONS, INC., Burlington, MA (US)

(72) Inventors: Murad Omar, Secaucus, NJ (US); Audrey Howell, Guilford, CT (US); Christine Zwart, Guilford, CT (US); Benjamin Horowitz, New Rochelle, NY (US); Tushar Parlikar, Somerville, MA (US); Ana Wu, White Plains, NY (US); John Peabody, JR., Hamden, CT (US)

(73) Assignee: BFLY OPERATIONS, INC., Burlington, MA (US)

(21) Appl. No.: 18/220,530

(22) Filed: Jul. 11, 2023

Related U.S. Application Data
(60) Provisional application No. 63/431,802, filed on Dec. 12, 2022.
(60) Provisional application No. 63/388,206, filed on Jul. 11, 2022.

(51) Int. Cl. G16H 40/67; A61B 8/00

(52) U.S. Cl. CPC G16H 40/67; A61B 8/4472; A61B 8/56; H04L 47/12

(57) ABSTRACT

An ultrasound system may include an ultrasound device that includes a first wired communication module and a first wireless communication module. The ultrasound system may further include a processing device that includes a second wired communication module and a second wireless communication module. The processing device may pair with the ultrasound device over a wireless connection using the first wireless communication module and the second wireless communication module. The processing device may further indicate a wireless connection on a display screen of the processing device. The processing device may further conduct an ultrasound imaging session over the wireless connection. The processing device may further determine that a cable has been connected between the ultrasound device and the processing device. The processing device may further terminate the wireless connection with the ultrasound device. The processing device may further indicate a wired connection on the display screen of the processing device.

### CROSS-REFERENCE TO RELATED APPLICATIONS

This United States Patent Application incorporates herein by reference U.S. Provisional Patent Application Ser. No. 63/388,206, titled “METHOD AND SYSTEM FOR WIRED AND WIRELESS COMMUNICATION USING ULTRASOUND DEVICES,” which was filed on Jul. 11, 2022, and U.S. Provisional Patent Application Ser. No. 63/431,802, titled “METHOD AND SYSTEM FOR WIRED AND WIRELESS COMMUNICATION USING ULTRASOUND DEVICES,” which was filed on Dec. 12, 2022.

### BACKGROUND

Imaging technologies are used for multiple purposes. One purpose is to non-invasively diagnose patients. Another purpose is to monitor the performance of medical procedures, such as surgical procedures. Yet another purpose is to monitor post-treatment progress or recovery. Thus, medical imaging technology is used at various stages of medical care. The value of a given medical imaging technology depends on various factors. Such factors include the quality of the images produced, the speed at which the images can be produced, the accessibility of the technology to various types of patients and providers, the potential risks and side effects of the technology to the patient, the impact on patient comfort, and the cost of the technology. The ability to produce three dimensional images is also a consideration for some applications.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments relate to an ultrasound system that includes an ultrasound device including a first wired communication module and a first wireless communication module. The ultrasound system further includes a processing device that includes a second wired communication module and a second wireless communication module. The processing device is configured to perform a method that includes pairing with the ultrasound device over a wireless connection using the first wireless communication module and the second wireless communication module. The method further includes indicating a wireless connection on a display screen of the processing device. The method further includes conducting an ultrasound imaging session over the wireless connection. The method further includes determining that a cable has been connected between the ultrasound device and the processing device. The method further includes terminating the wireless connection with the ultrasound device. The method further includes indicating a wired connection on the display screen of the processing device. The method further includes continuing to conduct the ultrasound imaging session over the cable using the first wired communication module and the second wired communication module.

In general, in one aspect, embodiments relate to an ultrasound system that includes an ultrasound device that includes a first wired communication module and a first wireless communication module. The ultrasound system further includes a processing device that includes a second wired communication module and a second wireless communication module. The processing device is configured to perform a method that includes determining that a cable has been connected between the ultrasound device and the processing device. The method further includes indicating a wired connection on a display screen of the processing device. The method further includes conducting an ultrasound imaging session over the cable using the first wired communication module and the second wired communication module. The method further includes determining that the cable between the ultrasound device and the processing device has been disconnected. The method further includes pairing with the ultrasound device over a wireless connection. The method further includes indicating a wireless connection on a display screen of the processing device. The method further includes terminating the wireless connection with the ultrasound device. The method further includes continuing to conduct the ultrasound imaging session over the wireless connection using the first wireless communication module and the second wireless communication module.

In light of the structure and functions described above, embodiments of the invention may include respective means adapted to carry out various steps and functions defined above in accordance with one or more aspects and any one of the embodiments of one or more aspect described herein.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 shows an example system in accordance with one or more embodiments of the technology.

FIG. 2 shows a block diagram of an example ultrasound device in accordance with one or more embodiments of the technology.

FIGS. 3A and 3B show flowcharts in accordance with one or more embodiments of the technology.

FIG. 4 shows an example pill that includes an example ultrasound probe in accordance with one or more embodiments of the technology.

FIGS. 5A, 5B, 5C, 5D, 5E, 5F, and 5G show examples in accordance with one or more embodiments of the technology.

FIG. 6 shows a schematic block diagram of an example ultrasound system in accordance with one or more embodiments of the technology.

FIG. 7 shows an example handheld ultrasound probe in accordance with one or more embodiments of the technology.

FIG. 8 shows an example patch that includes an example ultrasound probe in accordance with one or more embodiments of the technology.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as by the use of the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

Some embodiments include systems and methods that include an ultrasound device capable of performing both wireless and wired connections to a processing device. In some situations, a wired connection may have advantages over a wireless connection. For example, with a wired connection, ultrasound imaging can start sooner because no wireless pairing is required. Likewise, a wired connection may operate at a cooler temperature than a wireless connection while also consuming less battery life. As another example, a wired connection may also have less risk of losing connectivity during an ultrasound scan. On the other hand, wireless functionality may provide several advantages over wired functionality. For example, some processing devices may lack an available wired connection port and thus a wireless connection may be the only available option for communicating with the ultrasound device. An ultrasound device may also require disinfecting to avoid cross-contamination with an imaged subject. By removing the necessity to be in close proximity with a processing device that receives ultrasound data, a wireless ultrasound device may better maintain a sterile environment and avoid contact with compromised surfaces. Some embodiments include systems and methods for switching from a wired connection to a wireless connection, and vice versa, between an ultrasound device and processing.

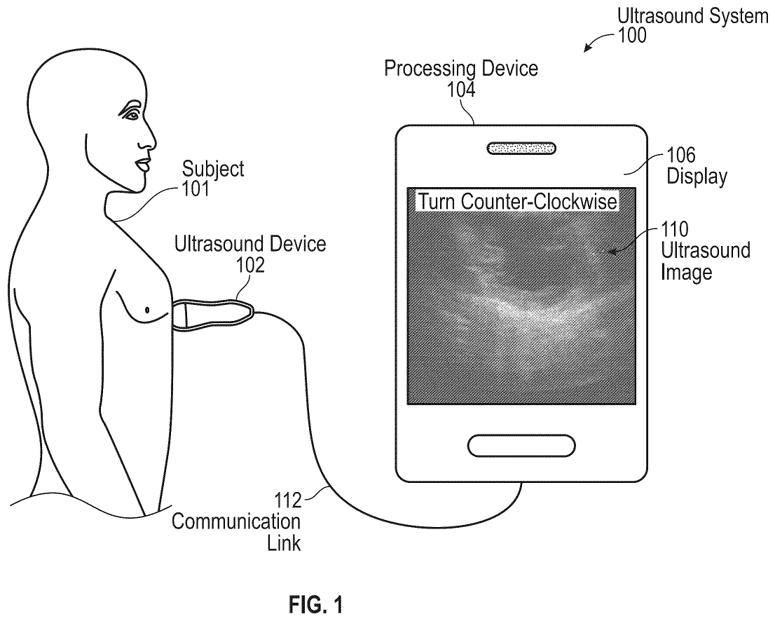

FIG. 1 shows an example ultrasound system 100 including an ultrasound device 102 configured to obtain an ultrasound image of a target anatomical view of a subject 101. As shown, the ultrasound system 100 comprises an ultrasound device 102 that is communicatively coupled to the processing device 104 by a communication link 112. The processing device 104 may be configured to receive ultrasound data from the ultrasound device 102 and use the received ultrasound data to generate an ultrasound image 110 on a display (which may be touch-sensitive) of the processing device 104. In some embodiments, the processing device 104 provides the operator with instructions (e.g., images, videos, or text) prior to the operator scanning the subject 101. The processing device 104 may provide quality indicators and/or labels of anatomical features during scanning of the subject 101 to assist a user in collecting clinically relevant ultrasound images.

The ultrasound device 102 may be configured to generate ultrasound data. The ultrasound device 102 may be configured to generate ultrasound data by, for example, emitting acoustic waves into the subject 101 and detecting the reflected acoustic waves. The detected reflected acoustic wave may be analyzed to identify various properties of the tissues through which the acoustic wave traveled, such as a density of the tissue. The ultrasound device 102 may be implemented in any of a variety of ways. For example, the ultrasound device 102 may be implemented as a handheld device (as shown in FIG. 1) or as a patch that is coupled to patient using, for example, an adhesive.

The ultrasound device 102 may transmit ultrasound data to the processing device 104 using the communication link 112. The communication link 112 may be a wired or wireless communication link. In some embodiments, the communication link 112 may be implemented as a cable such as a Universal Serial Bus (USB) cable or a Lightning cable. In these embodiments, the cable may also be used to transfer power from the processing device 104 to the ultrasound device 102. In other embodiments, the communication link 112 may be a wireless communication link such as a BLUETOOTH, WiFi, or ZIGBEE wireless communication link.

The processing device 104 may comprise one or more processing elements (such as a processor) to, for example, process ultrasound data received from the ultrasound device 102. Additionally, the processing device 104 may comprise one or more storage elements (such as a non-transitory computer readable medium) to, for example, store instructions that may be executed by the processing element(s) and/or store all or any portion of the ultrasound data received from the ultrasound device 102. It should be appreciated that the processing device 104 may be implemented in any of a variety of ways. For example, the processing device 104 may be implemented as a mobile device (e.g., a mobile smartphone, a tablet, or a laptop) with an integrated display 106 as shown in FIG. 1. In other examples, the processing device 104 may be implemented as a stationary device such as a desktop computer.

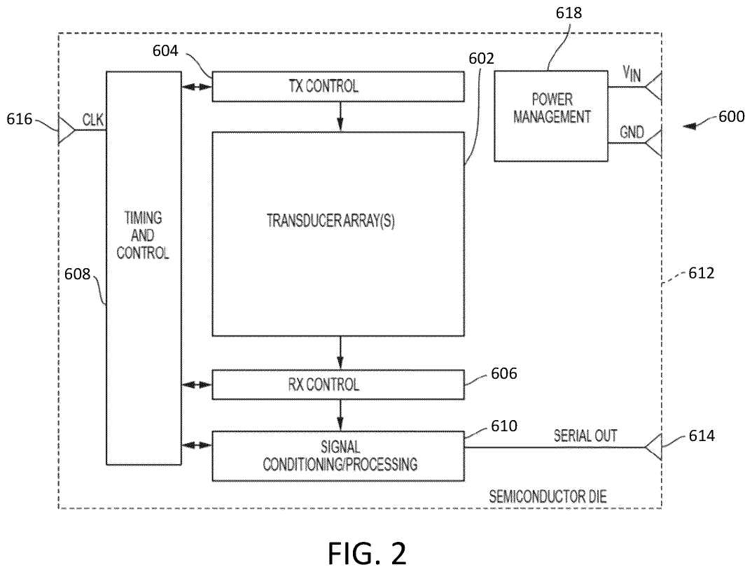

FIG. 2 is a block diagram of an example of an ultrasound device in accordance with some embodiments of the technology described herein. The illustrated ultrasound device 600 may include one or more ultrasonic transducer arrangements (e.g., arrays) 602, transmit (TX) circuitry 604, receive (RX) circuitry 606, a timing and control circuit 608, a signal conditioning/processing circuit 610, and/or a power management circuit 618.

The one or more ultrasonic transducer arrays 602 may take on any of numerous forms, and aspects of the present technology do not necessarily require the use of any particular type or arrangement of ultrasonic transducer cells or ultrasonic transducer elements. For example, multiple ultrasonic transducer elements in the ultrasonic transducer array 602 may be arranged in one-dimension, or two-dimensions. Although the term “array” is used in this description, it should be appreciated that in some embodiments the ultrasonic transducer elements may be organized in a non-array fashion. In various embodiments, each of the ultrasonic transducer elements in the array 602 may, for example, include one or more capacitive micromachined ultrasonic transducers (CMUTs), or one or more piezoelectric micromachined ultrasonic transducers (PMUTs).

In a non-limiting example, the ultrasonic transducer array 602 may include between approximately 6,000-10,000 (e.g., 8,960) active CMUTs on the chip, forming an array of hundreds of CMUTs by tens of CMUTs (e.g., 140×64). The CMUT element pitch may be between 150-250 um, such as 208 um, and thus, result in the total dimension of between 10-50 mm by 10-50 mm (e.g., 29.12 mm×13.312 mm).

In some embodiments, the TX circuitry 604 may, for example, generate pulses that drive the individual elements of, or one or more groups of elements within, the ultrasonic transducer array(s) 602 so as to generate acoustic signals to be used for imaging. The RX circuitry 606, on the other hand, may receive and process electronic signals generated by the individual elements of the ultrasonic transducer array(s) 602 when acoustic signals impinge upon such elements.

With further reference to FIG. 2, in some embodiments, the timing and control circuit 608 may be, for example, responsible for generating all timing and control signals that are used to synchronize and coordinate the operation of the other elements in the device 600. In the example shown, the timing and control circuit 608 is driven by a single clock signal CLK supplied to an input port 616. The clock signal CLK may be, for example, a high-frequency clock used to drive one or more of the on-chip circuit components. In some embodiments, the clock signal CLK may, for example, be a 1.5625 GHz or 2.5 GHz clock used to drive a high-speed serial output device (not shown in FIG. 2) in the signal conditioning/processing circuit 610, or a 20 Mhz or 40 MHz clock used to drive other digital components on the die 612, and the timing and control circuit 608 may divide or multiply the clock CLK, as necessary, to drive other components on the die 612. In other embodiments, two or more clocks of different frequencies (such as those referenced above) may be separately supplied to the timing and control circuit 608 from an off-chip source.

In some embodiments, the output range of a same (or single) transducer unit in an ultrasound device may be anywhere in a range of 1-12 MHz (including the entire frequency range from 1-12 MHz), making it a universal solution, in which there is no need to change the ultrasound heads or units for different operating ranges or to image at different depths within a patient. That is, the transmit and/or receive frequency of the transducers of the ultrasonic transducer array may be selected to be any frequency or range of frequencies within the range of 1 MHz-12 MHz. The universal device 600 described herein may thus be used for a broad range of medical imaging tasks including, but not limited to, imaging a patient's liver, kidney, heart, bladder, thyroid, carotid artery, lower venous extremity, and performing central line placement. Multiple conventional ultrasound probes would have to be used to perform all these imaging tasks. By contrast, a single universal ultrasound device 600 may be used to perform all these tasks by operating, for each task, at a frequency range appropriate for the task, as shown in the examples of Table 1 together with corresponding depths at which the subject may be imaged.

TABLE 1
Illustrative depths and frequencies at
which an ultrasound device implemented in accordance
with embodiments described herein may image a subject.

| Organ | Frequencies | Depth (up to) |
|---|---|---|
| Liver/Right Kidney | 2-5 MHz | 15-20 cm |
| Cardiac (adult) | 1-5 MHz | 20 cm |
| Bladder | 2-5 MHz; 3-6 MHz | 10-15 cm; 5-10 cm |
| Lower extremity venous | 4-7 MHz | 4-6 cm |
| Thyroid | 7-12 MHz | 4 cm |
| Carotid | 5-10 MHz | 4 cm |
| Central Line Placement | 5-10 MHz | 4 cm |

The power management circuit 618 may be, for example, responsible for converting one or more input voltages VIN from an off-chip source into voltages needed to carry out operation of the chip, and for otherwise managing power consumption within the device 600. In some embodiments, for example, a single voltage (e.g., 12V, 80V, 100V, 120V, etc.) may be supplied to the chip and the power management circuit 618 may step that voltage up or down, as necessary, using a charge pump circuit or via some other DC-to-DC voltage conversion mechanism. In other embodiments, multiple different voltages may be supplied separately to the power management circuit 618 for processing and/or distribution to the other on-chip components.

In the embodiment shown above, all of the illustrated elements are formed on a single semiconductor die 612. It should be appreciated, however, that in alternative embodiments one or more of the illustrated elements may be instead located off-chip, in a separate semiconductor die, or in a separate device. Alternatively, one or more of these components may be implemented in a DSP chip, a field programmable gate array (FPGA) in a separate chip, or a separate application specific integrated circuitry (ASIC) chip. Additionally, and/or alternatively, one or more of the components in the beamformer may be implemented in the semiconductor die 612, whereas other components in the beamformer may be implemented in an external processing device in hardware or software, where the external processing device is capable of communicating with the ultrasound device 600.

In addition, although the illustrated example shows both TX circuitry 604 and RX circuitry 606, in alternative embodiments only TX circuitry or only RX circuitry may be employed. For example, such embodiments may be employed in a circumstance where one or more transmission-only devices are used to transmit acoustic signals and one or more reception-only devices are used to receive acoustic signals that have been transmitted through or reflected off of a subject being ultrasonically imaged.

It should be appreciated that communication between one or more of the illustrated components may be performed in any of numerous ways. In some embodiments, for example, one or more high-speed busses (not shown), such as that employed by a unified Northbridge, may be used to allow high-speed intra-chip communication or communication with one or more off-chip components.

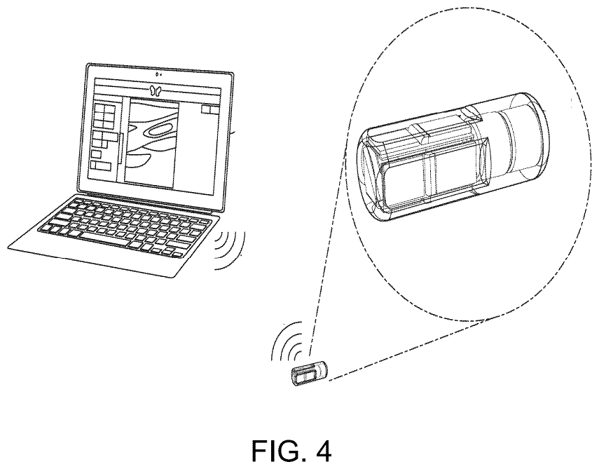

In some embodiments, the ultrasonic transducer elements of the ultrasonic transducer array 602 may be formed on the same chip as the electronics of the TX circuitry 604 and/or RX circuitry 606. The ultrasonic transducer arrays 602, TX circuitry 604, and RX circuitry 606 may be, in some embodiments, integrated in a single ultrasound probe. In some embodiments, the single ultrasound probe may be a handheld probe including, but not limited to, the handheld probes described below with reference to FIG. 7. In other embodiments, the single ultrasound probe may be embodied in a patch that may be coupled to a patient. FIG. 8 provides a non-limiting illustration of such a patch. The patch may be configured to transmit, wirelessly, data collected by the patch to one or more external devices for further processing. In other embodiments, the single ultrasound probe may be embodied in a pill that may be swallowed by a patient. The pill may be configured to transmit, wirelessly, data collected by the ultrasound probe within the pill to one or more external devices for further processing. FIG. 4 illustrates a non-limiting example of such a pill.

A CMUT may include, for example, a cavity formed in a CMOS wafer, with a membrane overlying the cavity, and in some embodiments sealing the cavity. Electrodes may be provided to create an ultrasonic transducer cell from the covered cavity structure. The CMOS wafer may include integrated circuitry to which the ultrasonic transducer cell may be connected. The ultrasonic transducer cell and CMOS wafer may be monolithically integrated, thus forming an integrated ultrasonic transducer cell and integrated circuit on a single substrate (the CMOS wafer).

In the example shown, one or more output ports 614 may output a high-speed serial data stream generated by one or more components of the signal conditioning/processing circuit 610. Such data streams may be, for example, generated by one or more USB 3.0 modules, and/or one or more 10 GB, 40 GB, or 100 GB Ethernet modules, integrated on the die 612. It is appreciated that other communication protocols may be used for the output ports 614.

In some embodiments, the signal stream produced on output port 614 can be provided to a computer, tablet, or smartphone for the generation and/or display of two-dimensional, three-dimensional, and/or tomographic images. In some embodiments, the signal provided at the output port 614 may be ultrasound data provided by the one or more beamformer components or auto-correlation approximation circuitry, where the ultrasound data may be used by the computer (external to the ultrasound device) for displaying the ultrasound images. In embodiments in which image formation capabilities are incorporated in the signal conditioning/processing circuit 610, even relatively low-power devices, such as smartphones or tablets which have only a limited amount of processing power and memory available for application execution, can display images using only a serial data stream from the output port 614. As noted above, the use of on-chip analog-to-digital conversion and a high-speed serial data link to offload a digital data stream is one of the features that helps facilitate an “ultrasound on a chip” solution according to some embodiments of the technology described herein.

Devices 600 such as that shown in FIG. 2 may be used in various imaging and/or treatment (e.g., HIFU) applications, and the particular examples described herein should not be viewed as limiting. In one illustrative implementation, for example, an imaging device including an N×M planar or substantially planar array of CMUT elements may itself be used to acquire an ultrasound image of a subject (e.g., a person's abdomen) by energizing some or all of the elements in the ultrasonic transducer array(s) 602 (either together or individually) during one or more transmit phases, and receiving and processing signals generated by some or all of the elements in the ultrasonic transducer array(s) 602 during one or more receive phases, such that during each receive phase the CMUT elements sense acoustic signals reflected by the subject. In other implementations, some of the elements in the ultrasonic transducer array(s) 602 may be used only to transmit acoustic signals and other elements in the same ultrasonic transducer array(s) 602 may be simultaneously used only to receive acoustic signals. Moreover, in some implementations, a single imaging device may include a P×Q array of individual devices, or a P×Q array of individual N×M planar arrays of CMUT elements, which components can be operated in parallel, sequentially, or according to some other timing scheme so as to allow data to be accumulated from a larger number of CMUT elements than can be embodied in a single device 600 or on a single die 612.

FIG. 6 illustrates a schematic block diagram of an example ultrasound system 700 which may implement various aspects of the technology described herein. In some embodiments, ultrasound system 700 may include an ultrasound device 702, an example of which is implemented in ultrasound device 600. For example, the ultrasound device 702 may be a handheld ultrasound probe. Additionally, the ultrasound system 700 may include a processing device 704, a communication network 716, and one or more servers 734. The ultrasound device 702 may be configured to generate ultrasound data that may be employed to generate an ultrasound image. The ultrasound device 702 may be constructed in any of a variety of ways. In some embodiments, the ultrasound device 702 includes a transmitter that transmits a signal to a transmit beamformer which in turn drives transducer elements within a transducer array to emit pulsed ultrasound signals into a structure, such as a patient. The pulsed ultrasound signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data. In some embodiments, the ultrasound device 702 may include an ultrasound circuitry 709 that may be configured to generate the ultrasound data. For example, the ultrasound device 702 may include semiconductor die 612 for implementing the various techniques described in.

Reference is now made to the processing device 704. In some embodiments, the processing device 704 may be communicatively coupled to the ultrasound device 702 (e.g., 102 in FIG. 1) wirelessly or in a wired fashion (e.g., by a detachable cord or cable) to implement at least a portion of the process for approximating the auto-correlation of ultrasound signals. In particular, the ultrasound device 702 may include a wired communication module 742 and a wireless communication module 744 for communicating with the processing device 704. The wired communication module 742 in the ultrasound device 702 may form a wired connection with a wired communication module 746 in the processing device 704. Likewise, the wireless communication module 744 in the ultrasound device 702 may form a wireless connection with a wireless communication module 748 in the processing device 704. A wired communication module may be a USB or Lightning communication module, while a wireless communication module may be a WiFi or Bluetooth communication module, etc.

Furthermore, one or more beamformer components may be implemented on the processing device 704. In some embodiments, the processing device 704 may include one or more processing devices (processors) 710, which may include specially-programmed and/or special-purpose hardware such as an ASIC chip. The processor 710 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network.

In some embodiments, the processing device 704 may be configured to process the ultrasound data received from the ultrasound device 702 to generate ultrasound images for display on the display screen 708. The processing may be performed by, for example, the processor(s) 710. The processor(s) 710 may also be adapted to control the acquisition of ultrasound data with the ultrasound device 702. The ultrasound data may be processed in real-time during a scanning session as the echo signals are received. In some embodiments, the displayed ultrasound image may be updated at a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

In some embodiments, the processing device 704 may be configured to perform various ultrasound operations using the processor(s) 710 (e.g., one or more computer hardware processors) and one or more articles of manufacture that include non-transitory computer-readable storage media such as the memory 712. The processor(s) 710 may control writing data to and reading data from the memory 712 in any suitable manner. To perform certain of the processes described herein, the processor(s) 710 may execute one or more processor-executable instructions stored in one or more non-transitory computer-readable storage media (e.g., the memory 712), which may serve as non-transitory computer-readable storage media storing processor-executable instructions for execution by the processor(s) 710.

The camera 720 may be configured to detect light (e.g., visible light) to form an image. The camera 720 may be on the same face of the processing device 704 as the display screen 708. The display screen 708 may be configured to display images and/or videos, and may be, for example, a liquid crystal display (LCD), a plasma display, and/or an organic light emitting diode (OLED) display on the processing device 704. The input device 718 may include one or more devices capable of receiving input from a user and transmitting the input to the processor(s) 710. For example, the input device 718 may include a keyboard, a mouse, a microphone, touch-enabled sensors on the display screen 708, and/or a microphone. The display screen 708, the input device 718, the camera 720, and/or other input/output interfaces (e.g., speaker) may be communicatively coupled to the processor(s) 710 and/or under the control of the processor 710.

It should be appreciated that the processing device 704 may be implemented in any of a variety of ways. For example, the processing device 704 may be implemented as a handheld device such as a mobile smartphone or a tablet. Thereby, a user of the ultrasound device 702 may be able to operate the ultrasound device 702 with one hand and hold the processing device 704 with another hand. In other examples, the processing device 704 may be implemented as a portable device that is not a handheld device, such as a laptop. In yet other examples, the processing device 704 may be implemented as a stationary device such as a desktop computer. The processing device 704 may be connected to the network 716 over a wired connection using a wired communication module 746 (e.g., via an Ethernet cable) and/or a wireless connection using a wireless communication module 748 (e.g., over a WiFi network). The processing device 704 may thereby communicate with (e.g., transmit data to or receive data from) the one or more servers 734 over the network 716. For example, a party may provide from the server 734 to the processing device 704 processor-executable instructions for storing in one or more non-transitory computer-readable storage media (e.g., the memory 712) which, when executed, may cause the processing device 704 to perform ultrasound processes. FIG. 6 should be understood to be non-limiting. For example, the ultrasound system 700 may include fewer or more components than shown and the processing device 704 and ultrasound device 702 may include fewer or more components than shown. In some embodiments, the processing device 704 may be part of the ultrasound device 702.

FIG. 7 illustrates an example handheld ultrasound probe, in accordance with certain embodiments described herein. The handheld ultrasound probe 780 may implement any of the ultrasound imaging devices described herein. The handheld ultrasound probe 780 may have a suitable dimension and weight. For example, the ultrasound probe 780 may have a cable for wired communication with a processing device, and have a length L about 100 mm-300 mm (e.g., 175 mm) and a weight about 200 grams-500 grams (e.g., 312 g). In another example, the ultrasound probe 780 may be capable of communicating with a processing device wirelessly. As such, the handheld ultrasound probe 780 may have a length about 140 mm and a weight about 265 g. It is appreciated that other dimensions and weight may be possible.

Further description of ultrasound devices and systems may be found in U.S. Pat. No. 9,521,991, the content of which is incorporated by reference herein in its entirety; and U.S. Pat. No. 11,311,274, the content of which is incorporated by reference herein in its entirety.

The following describes various methods whereby a processing device (e.g., a smartphone or a tablet) may be wirelessly paired with an ultrasound device. Both the processing device and the ultrasound device may include wireless modules configured to communicate over wireless connections. In some embodiments, the wireless connection may be a WiFi connection. In other embodiments, the wireless connection may be a Bluetooth or Zigbee connection. The wireless connection may be used for transmitting commands (e.g., imaging parameters) from the processing device to the ultrasound device and for transmitting ultrasound data from the ultrasound device to the processing device. The processing device may have an application (“app”) programmed onto it through which such communication is facilitated, and in which a user can view and analyze ultrasound images generated based on ultrasound data collected by the ultrasound device and transmitted to the processing device.

In some embodiments, the service set identifier (SSID) for the wireless connection may be the serial number of the ultrasound device, and the password for the wireless connection may be derived from the serial number. In some embodiments, the serial number for the ultrasound device may also be located on the ultrasound device. For example, the serial number may be located on a label affixed to the ultrasound device.

In some embodiments, a wireless connection is established using a matrix code, such as a QR (quick response) code or a Data Matrix code. In such embodiments, during pairing, the processing device may cause a camera window to open in the app along with a prompt asking the user to scan a matrix code located on the ultrasound device. For example, the matrix code may be located on a label affixed to the ultrasound device. In some embodiments, the data matrix may encode the serial number of the probe. In some embodiments, the processing device may prompt the user to press and hold (e.g., for 2 seconds) a button on the ultrasound device, which may turn on the wireless communication module in the ultrasound device, thus allowing a wireless connection to be established. The processing device may capture an image of the matrix code using its camera, process the image to determine the SSID and password, and establish the wireless connection (e.g., a WiFi connection) using the SSID and password.

In some embodiments, if the processing device is not successful in capturing an image of the matrix code and/or not successful in processing the image to determine the SSID and password, the processing device may prompt the user in the app to manually enter the serial number of the ultrasound device into the processing device (e.g., using a keyboard displayed on the processing device's display screen). The processing device may then use the serial number to determine the SSID and password, and establish the wireless connection using the SSID and password.

In some embodiments, a wireless connection is established using wireless transmission, for example, using Bluetooth. In such embodiments, the processing device may prompt the user in the app to press and hold (e.g., for 2 seconds) a button on the ultrasound device, which may turn on the wireless communication module in the ultrasound device, thus allowing a wireless connection to be established. Additionally, pressing the button may cause the ultrasound device to broadcast the SSID over Bluetooth. The processing device may read the broadcasted SSID, determine the password based on the SSID, and establish the wireless connection (e.g., WiFi) using the SSID and password.

In some embodiments, if the processing device is not successful in reading the broadcasted SSID and/or not successful in processing the broadcast to determine the SSID and password, the processing device may prompt the user in the app to manually enter the serial number of the ultrasound device into the processing device (e.g., using a keyboard displayed on the processing device's display screen). The processing device may then use the serial number to determine the SSID and password, and establish the wireless connection using the SSID and password. If the processing device detects multiple ultrasound devices broadcasting their SSIDs using Bluetooth, the processing device may select the ultrasound device broadcasting with the strongest signal.

When pairing is initiated, the processing device may be configured to choose the least congested channel for the wireless connection. Once the wireless connection is established, the processing device may initiate an imaging session in the app, whereby the user may select an ultrasound imaging mode, preset, and various other parameters, and the ultrasound device may begin to collect, process, and transmit ultrasound data to the processing device for further processing and display on the processing device as ultrasound images.

In some embodiments, during the pairing process, the processing device may prompt the user in the app whether to auto connect next time. If the user selects the auto connect option, at a future time, the processing device may automatically scan for the ultrasound device that it was connected to last time. If this ultrasound device is on and not connected to another processing device, the processing device may automatically present the user with a prompt whether to connect to it. If this ultrasound device is not on or is connected to another processing device, the processing device may prompt the user to initiate pairing to a different ultrasound device (e.g., using a matrix code or Bluetooth as described above).

In some embodiments, the processing device may present the user in the app with a list of a certain number (e.g., 5) of the last ultrasound devices to which the ultrasound device connected and prompt the user to select one. The processing device may then attempt to pair to the selected ultrasound device.

The following describes various methods whereby a processing device (e.g., a smartphone or a tablet) that was previously wirelessly paired with an ultrasound device may be disconnected from the ultrasound device. Upon disconnecting, the app may connect back to the wireless network to which the processing device was previously connected (e.g., the wireless network for the medical institution where the ultrasound device is located, or a different home network) and may upload any ultrasound studies (i.e., ultrasound images, cines, and/or worksheets) that were not yet uploaded to a server.

In some embodiments, the processing device may automatically disconnect from the ultrasound device. In some embodiments, if a cable (e.g., a USB or Lightning cable over which the ultrasound device and processing device can communicate) is connected between the ultrasound device and the processing device, the processing device may automatically disconnect from its wireless connection with the ultrasound device. In some embodiments, if the app has been backgrounded on the processing device for more than a certain number of seconds (e.g., 10), the processing device may automatically disconnect from its wireless connection with the ultrasound device. In some embodiments, if the app has been killed on the processing device, the processing device may automatically disconnect from its wireless connection with the ultrasound device. In some embodiments, if the user has navigated in the app away from an exam screen to a different screen not requiring communication between the ultrasound device and processing device (e.g., a patient editing screen), the processing device may automatically disconnect from its wireless connection with the ultrasound device. In some embodiments, if the wireless connection between the processing device and the ultrasound device becomes weak or unstable for a certain number of seconds (e.g., 10), the processing device may automatically disconnect from its wireless connection with the ultrasound device.

In some embodiments, a user may select an option in the app to manually disconnect the processing device from its wireless connection with the ultrasound device, in response to which the processing device may disconnect from its wireless connection with the ultrasound device. In some embodiments, in response to a user pressing a button on the ultrasound device for a certain amount of time (e.g., 5 seconds), the ultrasound device may power down and the wireless connection may be terminated.

In some embodiments, if the ultrasound device and processing device are connected with a wireless connection, but no active scanning is occurring, then after a certain amount of time the processing device may prompt the user whether to continue the wireless connection. If the user selects to disconnect the wireless connection, or does not make a selection within a certain amount of time, the processing device may disconnect from its wireless connection with the ultrasound device.

In some embodiments, if the ultrasound device and processing device are connected with a wireless connection, but the processing device detects that the connection strength is weak, then the processing device may prompt the user to move the processing device closer to the ultrasound device, or to connect the processing device and ultrasound device with a wired connection.

In some embodiments, if the ultrasound device and processing device are connected with a wireless connection, but the processing device detects that the wireless connection is unstable (e.g., multiple packets are dropped per frame) for a certain amount of time, then the processing device may rescan for a less congested channel. If the processing device does not find a less congested channel, the processing device may prompt the user to connect the processing device and ultrasound device with a wired connection, or to try reconnecting the processing device and ultrasound device with a wireless connection.

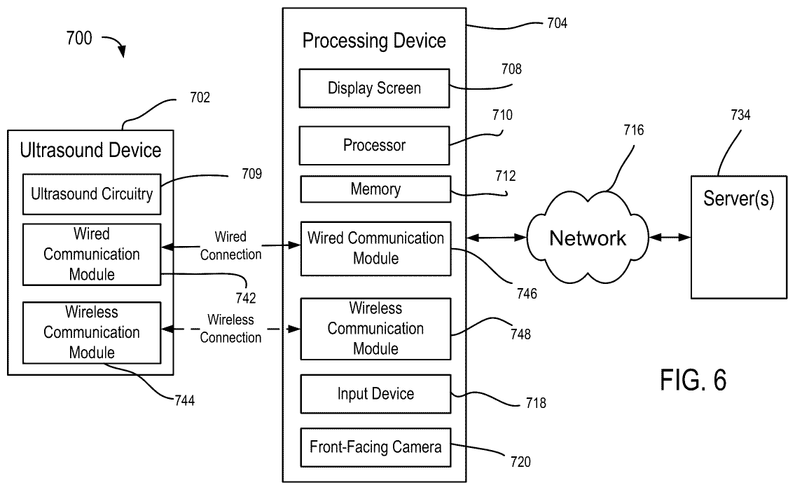

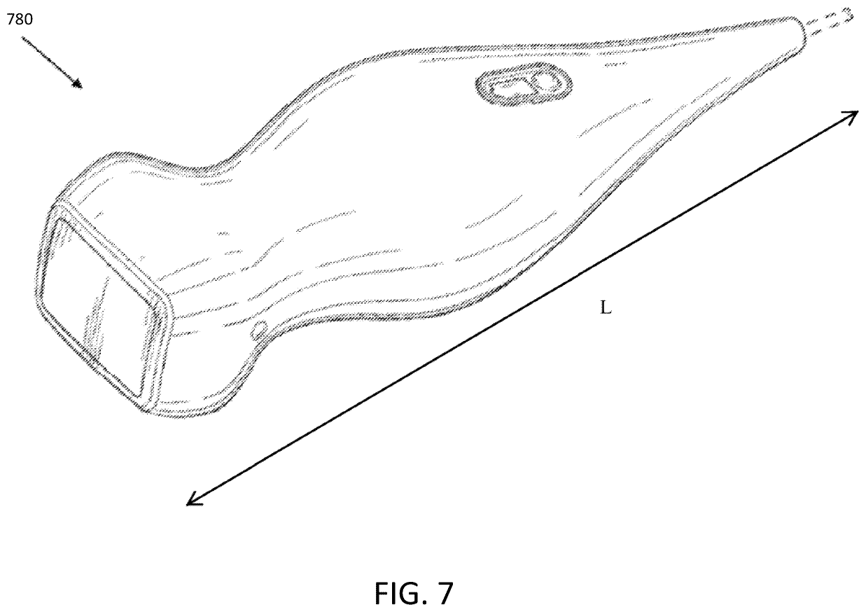

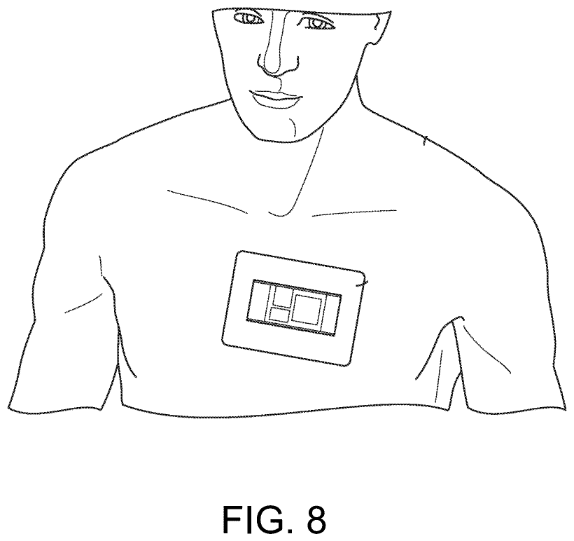

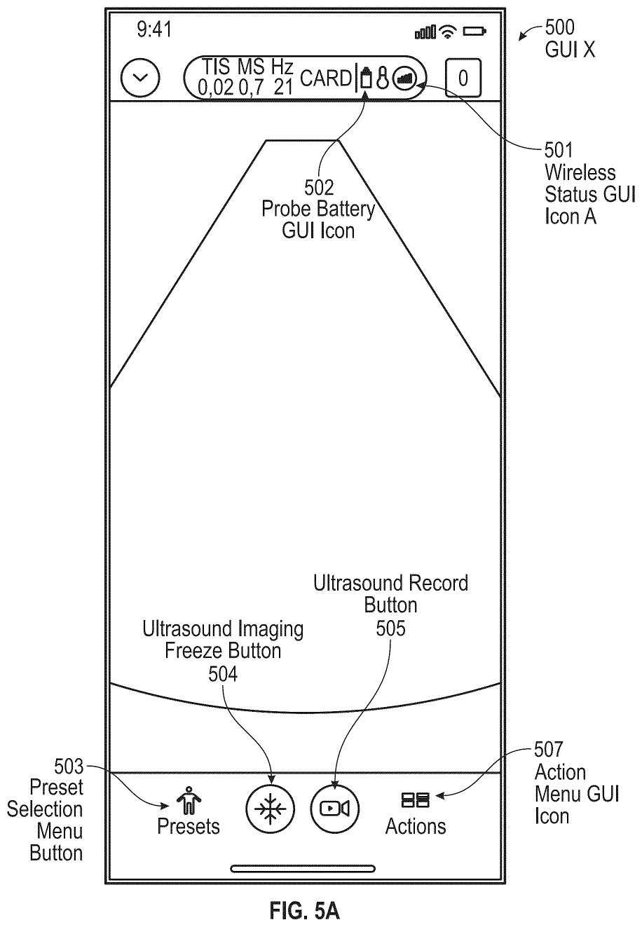

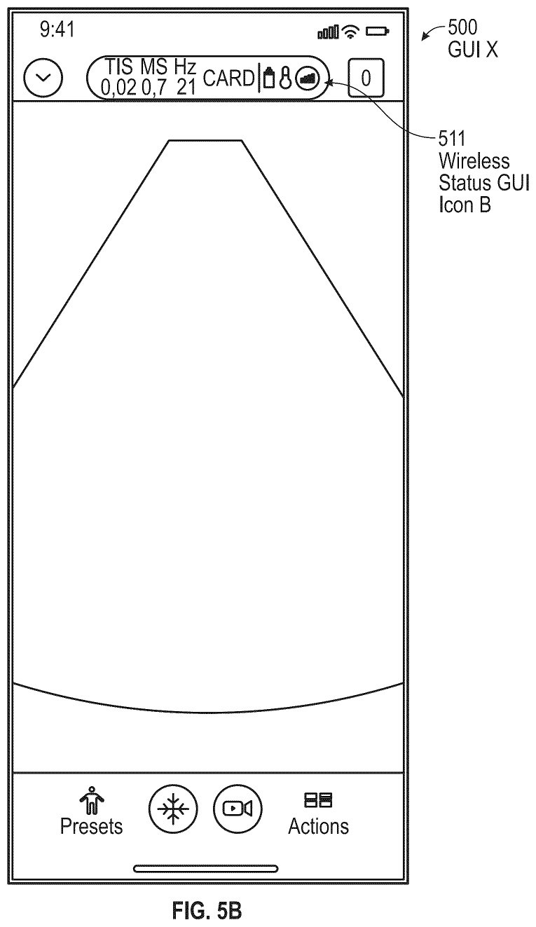

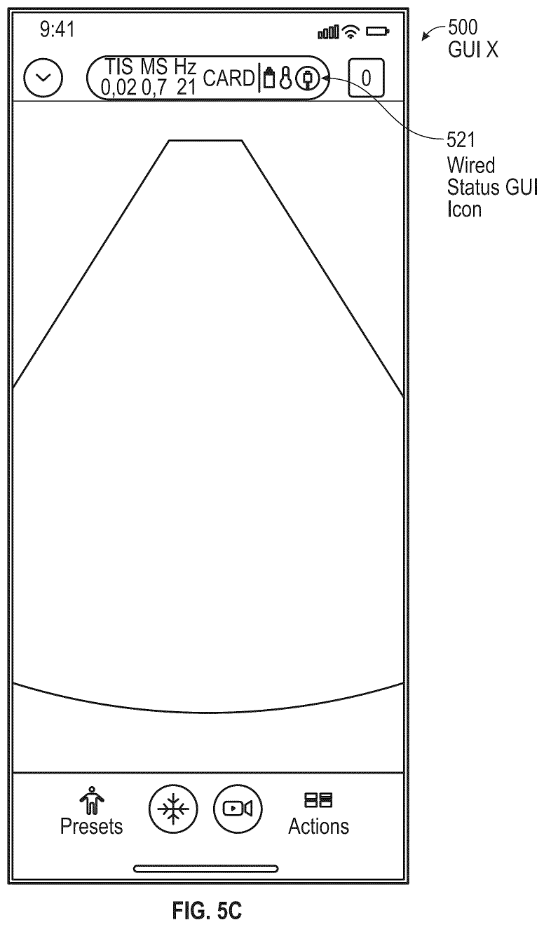

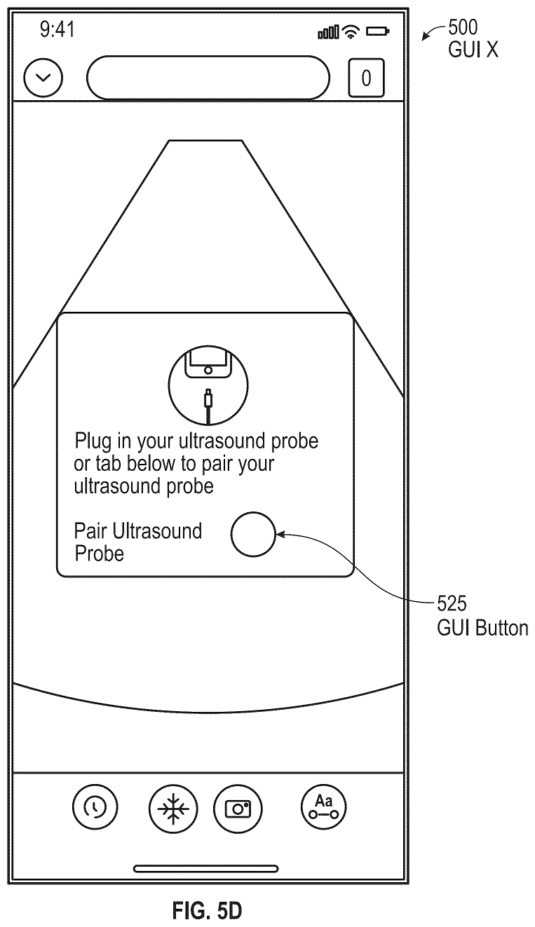

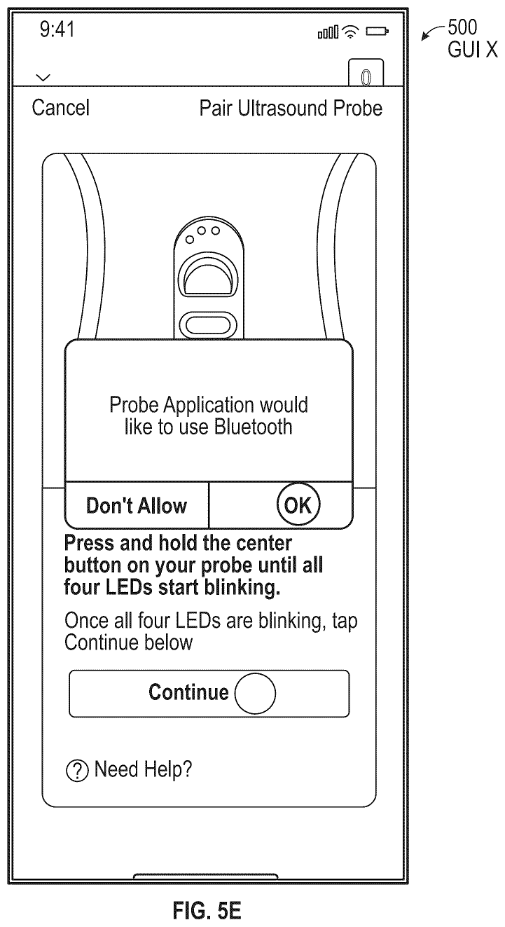

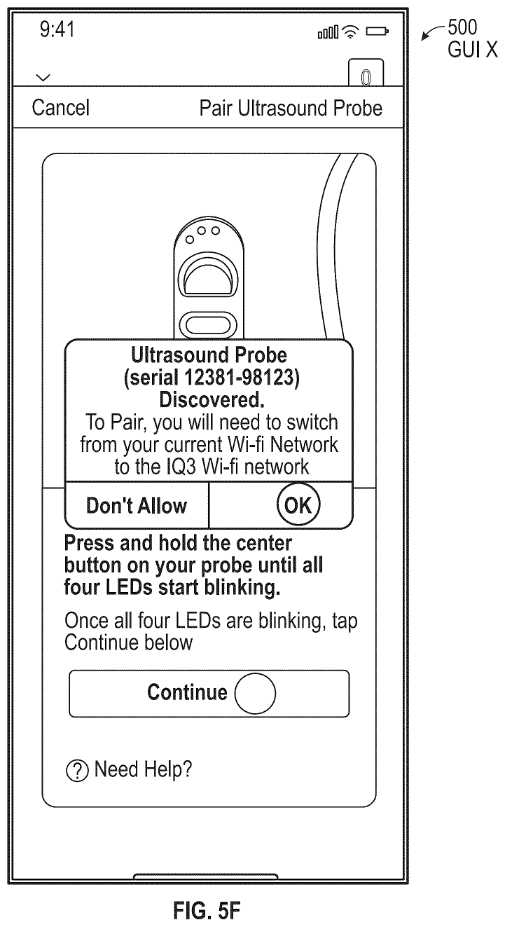

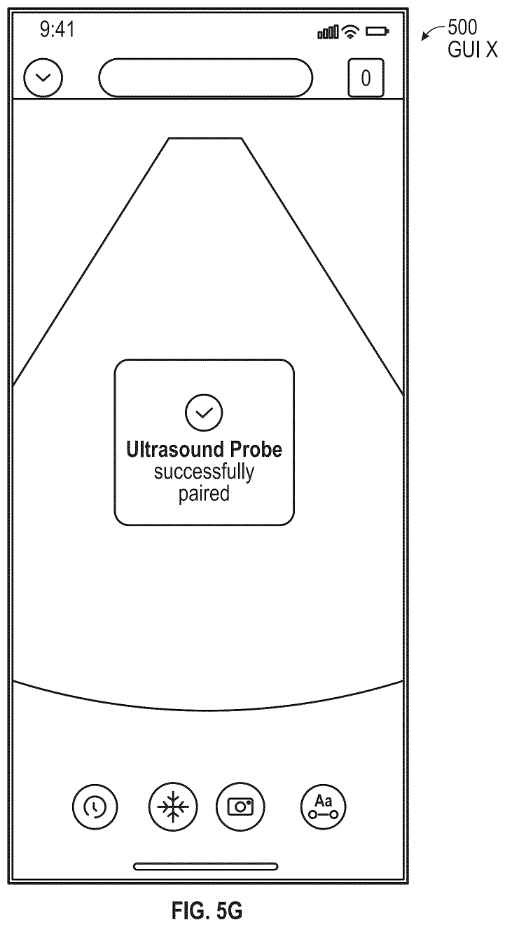

Turning to FIGS. 5A, 5B, 5C, 5D, 5E, 5F, and 5G, FIGS. 5A-5G provide examples of graphical user interfaces (GUIs) for managing wireless and wired connections between an ultrasound probe and a processing device in accordance with one or more embodiments. The following examples are for explanatory purposes only and not intended to limit the scope of the disclosed technology.

In FIG. 5A, a graphical user interface (GUI) X 500 is shown operating on a processing device (not shown) with various graphical icons, i.e., a wireless status GUI icon A 501, a probe battery GUI icon 502, an action menu GUI icon 507, and various GUI buttons, i.e., preset selection menu button 503, ultrasound imaging freeze button 504 and an ultrasound record button 505. For example, the GUI X 500 may be presented on a touchscreen where a user may perform various user selections using different GUI buttons. When a user contacts a GUI button with an input object (e.g., a finger or a stylus), one or more touch sensors may indicate the user selection. As shown in FIG. 5A, a wireless connection is indicated as being operational between an ultrasound probe (not shown) and the processing device by the wireless status GUI icon A 501. Likewise, the probe battery GUI icon 502 indicates that the ultrasound probe is near a fully-charged battery.

FIG. 5B shows that the GUI X 500 has changed to a different wireless status graphical icon, i.e., wireless status GUI icon B 511. The wireless status GUI icon B 511 indicates that a weak wireless connection exists between the ultrasound probe and the processing device.

FIG. 5C shows another GUI icon, i.e., a wired status GUI icon 521, indicating that a wired connection exists between the ultrasound probe and the processing device.

Turning to FIGS. 5D, 5E, 5F, and 5G, FIGS. 5D-5G show an example pairing between the ultrasound probe and a processing device. In FIG. 5D, the GUI X 500 provides a message to connect the ultrasound probe to processing device using a cable or select a wireless pairing to form a wireless network connection. In FIG. 5E, a user makes a selection (e.g., tapping a GUI Button 525) to enable Bluetooth access on the processing device for a software program communicating with the ultrasound probe. Accordingly, the ultrasound device and the processing device may establish a wireless network connection using a Bluetooth protocol. In FIG. 5F, a broadcast message is discovered that identifies a WiFi network address, which is subsequently used to establish a wireless network connection over a WiFi protocol. In FIG. 5G, a message is shown in GUI X 500 indicating that a successful pairing has occurred between the ultrasound probe and the processing device.

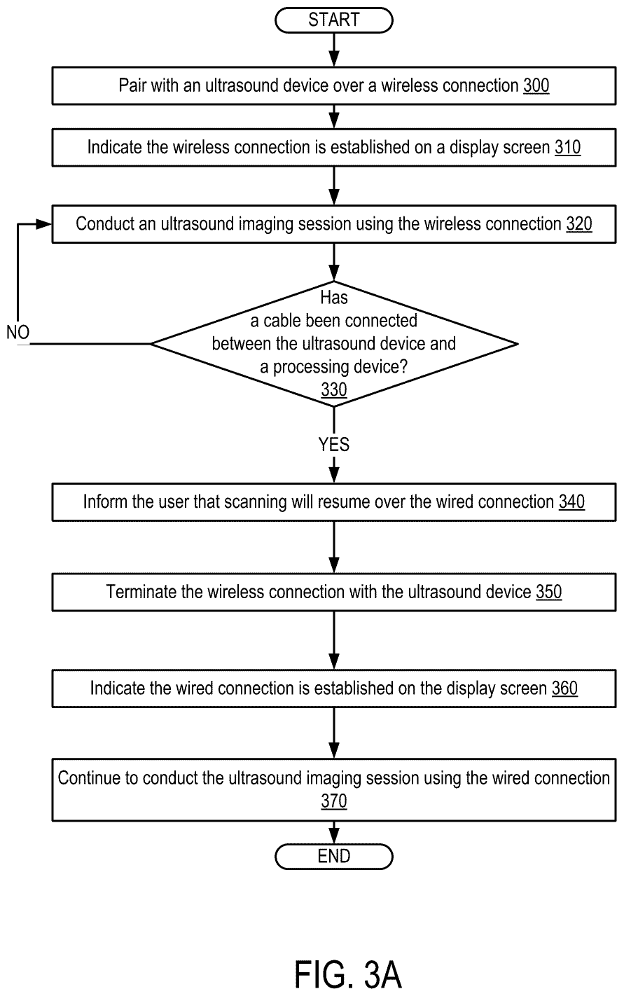

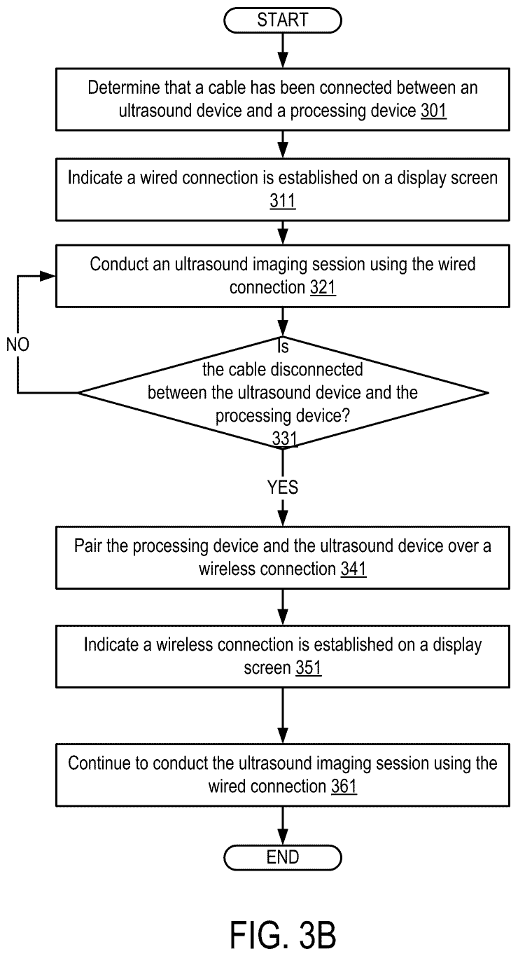

Turning to FIGS. 3A and 3B, FIGS. 3A and 3B show flowcharts in accordance with one or more embodiments. Specifically, FIGS. 3A and 3B describe methods for managing wireless and wired connections between an ultrasound device and a processing device. The blocks in FIGS. 3A and 3B may be performed by a processing device (e.g., the processing device 104). The processing device may be capable of communicating with an ultrasound device (e.g., the ultrasound device 102) both over a wireless connection and a wired connection. Each of the processing device and the ultrasound device may have a wireless communication module and a wired communication module facilitating wireless and wired connections, respectively, between the two devices. While the various blocks in FIGS. 3A and 3B are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

Turning to FIG. 3A, in Block 300, the processing device pairs with the ultrasound device over a wireless connection in accordance with one or more embodiments. Any of the methods listed above for pairing a processing device and an ultrasound device may be used.

In Block 310, the processing device indicates a wireless connection is present on a display screen in accordance with one or more embodiments. For example, the processing device may display the GUI icon A 501 or the GUI icon B 501 based on a strength of the wireless connection.

In Block 320, the processing device conducts an ultrasound imaging session using the wireless connection. Conducting the ultrasound imaging session using the wireless connection may include the processing device transmitting one or more commands to the ultrasound device over the wireless connection in accordance with one or more embodiments. For example, commands may correspond to various messages that adjust one or more settings on the ultrasound device, e.g., changing ultrasound parameters or presets. Likewise, commands may also include various operation requests, e.g., a request to begin performing an ultrasound operation or to shut down the ultrasound device. Commands may also be used to obtain ultrasound data from the ultrasound device over the wireless connection, e.g., a request to offload any stored ultrasound data. Conducting the ultrasound imaging session using the wireless connection may also include the processing device obtaining ultrasound data from the ultrasound device over the wireless connection.

In Block 330, the processing device determines whether a cable (e.g., a USB or Lightning cable over which the ultrasound device and processing device can communicate) has been connected between the ultrasound device and the processing device. If a cable has not been connected between the ultrasound device and the processing device, proceed to Block 320. If a cable has been connected between the ultrasound device and the processing device, proceed to Block 340.

In Block 340, the processing device informs the user (e.g., by displaying text on its display screen) that scanning will resume over the wired connection. In some embodiments, Block 340 may be omitted.

In Block 350, the processing device terminates the wireless connection with the ultrasound device. In some embodiments, prior to terminating the wireless connection, the processing device may transmit commands to the ultrasound device, over the wireless connection, to turn off its wireless communication module. In some embodiments, the ultrasound device itself may determine that the cable has been connected between the ultrasound device and the processing device and turn off its wireless communication module based on that determination.

In Block 360, the processing device indicates a wired connection is established on the display screen in accordance with one or more embodiments. For example, the processing device may display the GUI icon 521.

In Block 370, the processing device continues to conduct the ultrasound imaging session using the wired connection (i.e., over the cable). Conducting the ultrasound imaging session using the wired connection may include the processing device transmitting one or more commands to the ultrasound device over the wired connection in accordance with one or more embodiments. For example, commands may correspond to various messages that adjust one or more settings on the ultrasound device, e.g., changing ultrasound parameters and/or presets. Likewise, commands may also include various operation requests, e.g., a request to begin performing an ultrasound operation or to shut down the ultrasound device. Commands may also be used to obtain ultrasound data from the ultrasound device over the wired connection, e.g., a request to offload any stored ultrasound data. Conducting the ultrasound imaging session using the wired connection may also include the processing device obtaining ultrasound data from the ultrasound device over the wired connection. In some embodiments, the ultrasound imaging session may use the same parameters and/or presets that were used in Block 310. In such embodiments, the ultrasound device may have saved these parameters and/or presets, may still be configured with these parameters and/or presets, or the processing device may transmit these parameters and/or presets to the ultrasound device again.

Turning to FIG. 3B, in Block 301, the processing device determines that a cable (e.g., a USB or Lightning cable over which the ultrasound device and processing device can communicate) has been connected between the ultrasound device and the processing device.

In Block 311, the processing device indicates a wired connection on its display screen in accordance with one or more embodiments. For example, the processing device may display the GUI icon 521.

In Block 321, the processing device conducts an ultrasound imaging session using the wired connection (i.e., over the cable). Conducting the ultrasound imaging session using the wired connection may include the processing device transmitting one or more commands to the ultrasound device over the wired connection in accordance with one or more embodiments. For example, commands may correspond to various messages that adjust one or more settings on the ultrasound device, e.g., changing ultrasound parameters and/or presets. Likewise, commands may also include various operation requests, e.g., a request to begin performing an ultrasound operation or to shut down the ultrasound device. Commands may also be used to obtain ultrasound data from the ultrasound device over the wireless connection, e.g., a request to offload any stored ultrasound data. Conducting the ultrasound imaging session using the wired connection may also include the processing device obtaining ultrasound data from the ultrasound device over the wired.

In Block 331, the processing device determines whether the cable between the ultrasound device and the processing device has been disconnected. If the cable has not been disconnected, proceed to Block 321. If the cable has been disconnected, proceed to Block 341.

In Block 341, the processing device pairs with the ultrasound device over a wireless connection in accordance with one or more embodiments. In some embodiments, the user may manually initiate the pairing using any of the methods listed above for pairing a processing device and an ultrasound device may be used. In some embodiments, the processing device may automatically initiate pairing with the ultrasound device. In such embodiments, the processing device may have received information necessary to determine the SSID and password from the ultrasound device over the wired connection and/or such information may have already been stored on the processing device. In some embodiments, the processing device may prompt the user to confirm that the processing device should switch from using its current wireless network (e.g., the wireless network for the medical institution where the ultrasound device is located, or a different home network) to the wireless network for communicating with the ultrasound device. In some embodiments, this switch may occur automatically. In some embodiments, the ultrasound device's wireless communication module may have been off prior to Block 341, and the user may turn on it, for example by pressing a button on the ultrasound device. In some embodiments, the ultrasound device's wireless communication module may have been on prior to Block 341. For example, the ultrasound device may always turn on its wireless communication module during scanning, even when communicating with the processing device over a wired connection.

In Block 351, the processing device indicates a wireless connection is established on a display screen in accordance with one or more embodiments. For example, the processing device may display the GUI icon A 501 or the GUI icon B 501 based on a strength of the wireless connection.

In Block 361, the processing device continues to conduct the ultrasound imaging session using the wireless connection. Conducting the ultrasound imaging session using the wireless connection may include the processing device transmitting one or more commands to the ultrasound device over the wireless connection in accordance with one or more embodiments. For example, commands may correspond to various messages that adjust one or more settings on the ultrasound device, e.g., changing ultrasound parameters and/or presets. Likewise, commands may also include various operation requests, e.g., a request to begin performing an ultrasound operation or to shut down the ultrasound device. Commands may also be used to obtain ultrasound data from the ultrasound device over the wireless connection, e.g., a request to offload any stored ultrasound data. Conducting the ultrasound imaging session using the wireless connection may also include the processing device obtaining ultrasound data from the ultrasound device over the wireless connection. In some embodiments, the ultrasound imaging session may use the same parameters and/or presets that were used in Block 311. In such embodiments, the ultrasound device may have saved these parameters and/or presets, may still be configured with these parameters and/or presets, or the processing device may transmit these parameters and/or presets to the ultrasound device again.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed is:

1. An ultrasound system, comprising:
an ultrasound device comprising a first wired communication module and a first wireless communication module; and
a processing device comprising a second wired communication module and a second wireless communication module, and the processing device is configured to:
pair with the ultrasound device over a wireless connection using the first wireless communication module and the second wireless communication module;
indicate a wireless connection on a display screen of the processing device;
conduct an ultrasound imaging session over the wireless connection;
determine that a cable has been connected between the ultrasound device and the processing device;
terminate the wireless connection with the ultrasound device;
indicate a wired connection on the display screen of the processing device; and
continue to conduct the ultrasound imaging session over the cable using the first wired communication module and the second wired communication module.

2. The ultrasound system of claim 1,
wherein the wireless connection is indicated using a graphical user interface (GUI) icon that is displayed based on a strength of the wireless connection.

3. The ultrasound system of claim 1,
wherein the processing device is further configured to open a camera window in a graphical user interface, and
wherein the wireless connection is established using a matrix code that is scanned using the camera window.

4. The ultrasound system of claim 1,
wherein the processing device is further configured to transmit a command to the ultrasound device using the wired connection, and
wherein the command adjusts one or more settings on the ultrasound device for the ultrasound imaging session.

5. The ultrasound system of claim 1,
wherein the processing device is further configured to transmit a command to the ultrasound device using the wireless connection, and
wherein the command comprises a request to shut down the ultrasound device.

6. The ultrasound system of claim 1,
wherein the ultrasound device is further configured to turn on the first wireless communication module in response to a button being pressed on the ultrasound device.

7. The ultrasound system of claim 1,
wherein the first wireless communication module and the second wireless communication module are WiFi communication modules.

8. The ultrasound system of claim 1,
wherein the first wired communication module and the second wireless communication module are Universal Serial Bus (USB) communication modules.

9. An ultrasound system, comprising:
an ultrasound device comprising a first wired communication module and a first wireless communication module; and
a processing device comprising a second wired communication module and a second wireless communication module, and the processing device is configured to:
determine that a cable has been connected between the ultrasound device and the processing device;
indicate a wired connection on a display screen of the processing device;
conduct an ultrasound imaging session over the cable using the first wired communication module and the second wired communication module;
determine that the cable between the ultrasound device and the processing device has been disconnected;
pair with the ultrasound device over a wireless connection;
indicate a wireless connection on a display screen of the processing device;
terminate the wireless connection with the ultrasound device; and
continue to conduct the ultrasound imaging session over the wireless connection using the first wireless communication module and the second wireless communication module.

10. The ultrasound system of claim 9,
wherein the processing device is further configured to provide a prompt in a graphical user interface for a user, and
wherein the prompt is configured to confirm whether to switch from a first wireless network to a second wireless network for communicating with the ultrasound device.

11. The ultrasound system of claim 9,
wherein the processing device is further configured to switch automatically from a first wireless network to a second wireless network for communicating with the ultrasound device.

12. The ultrasound system of claim 9,
wherein the wired connection is indicated using a graphical user interface (GUI) icon.

13. The ultrasound system of claim 9,
wherein the processing device is further configured to transmit a command to the ultrasound device using the wired connection, and
wherein the command adjusts one or more settings on the ultrasound device for the ultrasound imaging session.

14. The ultrasound system of claim 9,
wherein the processing device is further configured to turn on the second wireless communication module while the ultrasound imaging session is being performed over the cable.

15. The ultrasound system of claim 9,
wherein the processing device is further configured to transmit a command to the ultrasound device using the wireless connection, and
wherein the command comprises a request to shut down the ultrasound device.

16. The ultrasound system of claim 9,
wherein the wireless connection is indicated using a graphical user interface (GUI) icon that is displayed based on a strength of the wireless connection.

17. The ultrasound system of claim 9,
wherein pairing the ultrasound device with the processing device is automatically initiated by the processing device.

18. The ultrasound system of claim 9,
wherein the processing device determines a least congested channel in a wireless network, and
wherein pairing the ultrasound device with the processing device is performed using the least congested channel.

19. The ultrasound system of claim 9,
wherein the first wireless communication module and the second wireless communication module are WiFi communication modules.

20. The ultrasound system of claim 9,
wherein the first wired communication module and the second wireless communication module are Universal Serial Bus (USB) communication modules.
